(19) United States
(12) Patent Application Publication
(10) Pub. No.: US 2020/0353236 A1
(43) Pub. Date: Nov. 12, 2020

(54) Multiply Sealed Access Port and Companion Devices

(71) Applicants: Andrew James Schwartz, Clyde Hill, WA (US); Richard Ubinas, Everett, WA (US)

(72) Inventors: Andrew James Schwartz, Clyde Hill, WA (US); Richard Ubinas, Everett, WA (US)

(73) Assignees: Andrew James Schwartz, Clyde Hill, WA (US); Richard Ubinas, Everett, WA (US)

(21) Appl. No.: 16/408,473

(22) Filed: May 10, 2019

(51) Int. Cl. A61M 39/02; A61M 5/168; A61M 5/172; A61M 39/04; A61M 39/08; G16H 20/17

(52) U.S. Cl. CPC A61M 39/0247; A61M 5/16886; A61M 5/172; A61M 39/04; A61M 2039/0054; G16H 20/17; A61M 2039/0205; A61M 2039/0258; A61M 2039/027; A61M 39/08

(57) ABSTRACT

An access port for transporting fluids into a patient's body utilizing a multiple seals in parallel is disclosed. The access port 100 includes a flanged base 102, a receptacle 106 with multiple seals 108 and 110, and a cannula 104. The access port may also include a guide needle for installation purposes, and a sanitary adaptor that seals the receptacle for long term usage. Adhesives or bandaging may utilize the flanged base to secure the access port to the patient. The flanged base may also be installed beneath the patient's skin so secure the access port. The receptacle utilizes a multitude of sealing mechanism, including but not limited to O-ring seals and screw-lock seals. The receptacle additionally contains and outlet flow path leading to the cannula. The cannula connects from the receptacle into the patient's body. The cannula is installed perpendicular to the plane of the flanged base and protrudes on the side of the access port facing into the patient's body while the receptacle faces outward. A number of devices may work in conjunction to access port 100 that interface with the multiple parallel seal mechanism. Guide needle 200 may be utilized during installation, sanitary seal 300 may be used for long term use, and band 500 in conjunction with bag 600 may be worn on a patient to deliver substances into their body. Programmable components 512, 606, and 712 may be implemented with these devices that enables a system that automatically identifies the substance being administered and dosing information, then relays that information to a doctor or caregiver.

### BACKGROUND—PRIOR ART

The following is a tabulation of some prior art that presently appears relevant:

U.S. Patents

| Patent Number | Kind Code | Issue Date | Patentee |
|---|---|---|---|
| 3,312,220 | A | 1967 Apr. 4 | Eisenberg |
| 3,375,310 | A | 1968 Mar. 26 | Koehn |
| 5,667,514 | A | 1997 Sep. 16 | Heller |
| 7,651,483 | B2 | 2010 Jan. 26 | Byrum et. al |
| 7,846,139 | B2 | 2010 Dec. 7 | Zinn et al. |
| 7,862,546 | B2 | 2011 Jan. 4 | Conlon et al. |
| 7,972,314 | B2 | 2011 Jul. 5 | Bizup et al. |
| 10/166,370 | B2 | 2019 Jan. 1 | Woehr et al. |

U.S. Patent Application Publications

| Patent Number | Kind Code | Publ. Date | Applicant |
|---|---|---|---|
| 20060135949 | A1 | 2006 Jun. 22 | Rome et al. |
| 20160000363 | A1 | 2016 Jan. 7 | Jones et al. |

### TECHNICAL FIELD

The present invention relates to medical access devices, and more specifically devices that provide a transdermal connection between a patients' body and an external source.

### BACKGROUND

Transdermal access to a patient's body has proven to be an integral part of the medical field. Many new therapies are designed based on the ability for chronic subcutaneous and intravenous access. Despite the high demand for this technology, fundamental problems have not been addressed by new technology. Venous catheters are perhaps the most common example of medical access technology. Venous catheters have issues with cleanliness and comfort that can lead to complications in patients. If a catheter is installed for an extended period is must be cleaned thoroughly between uses or an infection may arise. On the other hand, frequently installing new catheters may have a negative effect on the condition of a patient's veins. Catheters are also designed to work in conjunction with intravenous bags. The thin flexible tubing of these bags requires that the catheter is secured in place to the patient's body so that the connection remains intact throughout treatment. The bulkiness of the assembly required to secure a catheter in place can be discomforting to a patient. Furthermore, many catheters have a metal needle that is inserted into the patient's vein. The metal needle embedded in a patient's body can be discomforting and reduces the patient's mobility. This is especially discomforting for patients with a catheter installed for extended periods. The many issues of discomfort can reduce patient participation which in effect means that the therapy is less effective.

Another example of medical access technology are access ports. These are installed in a patient using an invasive surgery. They are often installed under the skin or secured with bolts and screws. They also utilize a cannula that is bored into a patients' central vein. These types of devices are designed for long term repeated use, however complications with surgery and infections may still arise. These devices are typically installed in elderly patients. There is no access port that is viable for younger people or children. These devices are designed to work in conjunction with intravenous bags and are like catheters at the point of treatment. In both conventional approaches to medical access technology, patients are bedridden during treatment and the same problem of discomfort and lack of mobility remain.

It would be more advantageous for a medical access device to address the needs of a non-invasive installation, have robust cleanliness design, enable new means of substance delivery, reduce bulk that limits mobility, and provide minimal patient discomfort.

### SUMMARY OF THE INVENTION

In accordance with one embodiment, a vascular access device, known as a ‘Button’, is installed onto the patient's skin. This Button is anchored to the patient by a flanged base wherein a narrow cannula is located on the inward facing side, and a receptacle with a multitude of seals in parallel on the outward facing side. The cannula provides access across the patient's skin, while the receptacle enables flow of substances between an external source and the Button. The flanged base is used in certain embodiments to secure the Button to the patient's body. The means to secure the flanged base includes but is not limited to adhesives and bandages.

In one embodiment for the subcutaneous access, the cannula is comprised of a rigid medically safe material, which includes but is not limited to stainless steel, titanium, other metals, and composite materials. In this embodiment the cannula provides the puncturing capacity to penetrate the patients skin.

In another embodiment for intravenous access, the cannula is comprised of a flexible medically safe material including but not limited to PTFE and silicone rubber. For installation a guide needle is inserted into the cannula. The guide needle may be composed of a material with sufficient puncturing capacity to penetrate the patient's skin.

Some embodiments of the invention may include sealing the receptacle with a sanitary seal. The seal comprising of material mimicking the Button. The seal will engage the doubly sealed mechanism of the receptacle to prevent air exposure while the device is not in use. The sanitary seal may further comprise of an adaptor on the outer facing side, compatible with external devices, such that the sanitary adaptor remains installed during use.

In accordance with another embodiment, an external storage device known as a ‘Band’, houses a substance container known as a ‘Bag’, connects to the receptacle of a Button to transport substances into a patient's body. The flow of substance from the Bag into the patient may be powered by a pump located on the Band. The Bag may be replaceable such that a single Band and Button may be used for a multitude of doses.

In one embodiment a programmable component is integrated into a device including but not limited to a ‘Bag’, ‘Band’ or ‘Button’. The programmable component may introduce the feature to identify the substance that is currently in place in the system. This component may additionally be used to verify that the correct substance with the proper dosage setting is applied. The programmable component may further be utilized for gathering data and exporting to an external databank for further analysis.

### BRIEF DESCRIPTION OF DRAWINGS

FIG. 1 is a top perspective view to a first embodiment of an access port;

FIG. 2 is a top view of the access port illustrated in FIG. 1;

FIG. 3 is a front elevation view of the access port illustrated in FIG. 1;

FIG. 4 is a bottom perspective view of the access port illustrated in FIG. 1;

FIG. 5 is a bottom view of the access port illustrated in FIG. 1;

FIG. 6 is a cross section of the access port of FIG. 1 taken along the lines A-A of FIG. 2 FIG. 7 is a top perspective view of an embodiment of a guide needle;

FIG. 8 is an exploded view of an embodiment of an assembly using the access port from FIG. 1 and the guide needle from FIG. 7;

FIG. 9 is a perspective view of the assembly illustrated in FIG. 8;

FIG. 10 is a front elevation view of the assembly illustrated in FIG. 8;

FIG. 11 is a cross section of the assembly of FIG. 8 taken along the lines B-B of FIG. 10;

FIG. 12 is a top perspective view of an embodiment of a sanitary adaptor;

FIG. 13 is a front elevation view of the sanitary adaptor from FIG. 12;

FIG. 14 is a bottom perspective view of the sanitary adaptor from FIG. 12;

FIG. 15 is an exploded view of an embodiment of an assembly using the access port from FIG. 1 and the sanitary adaptor from FIG. 12;

FIG. 16 is a perspective view of the assembly illustrated in FIG. 15;

FIG. 17 is a cross section of the assembly of FIG. 15 taken along the lines C-C of FIG. 16;

FIG. 18 is a top perspective view to a second embodiment of an access port;

FIG. 19 is a top view of the access port illustrated in FIG. 18;

FIG. 20 is a bottom view of the access port illustrated in FIG. 18;

FIG. 21 is a bottom perspective view of the access port illustrated in FIG. 18;

FIG. 22 is a front elevation view of the access port illustrated in FIG. 18;

FIG. 23 is a top perspective view to a first embodiment of an external storage device in an unfastened state;

FIG. 24 is an exploded view of an embodiment of an assembly using the external storage device from FIG. 23, and a first embodiment of a substance container;

FIG. 25 is a cross section of the assembly of FIG. 24 taken along the lines D-D of FIG. 23;

FIG. 26 is a magnified view of the cross section of FIG. 25;

FIG. 27 is a top perspective view of the external storage device illustrated in FIG. 23 in a fastened state;

FIG. 28 is a front elevation view of the external storage device illustrated in FIG. 26;

FIG. 29 is a front elevation view of an assembly of the access port of FIG. 1 and the external storage device illustrated in FIG. 26;

FIG. 30 is a perspective view of the installation of the access port of FIG. 1 onto a patient, with the access port illustrated in this instance installed on the patients arm;

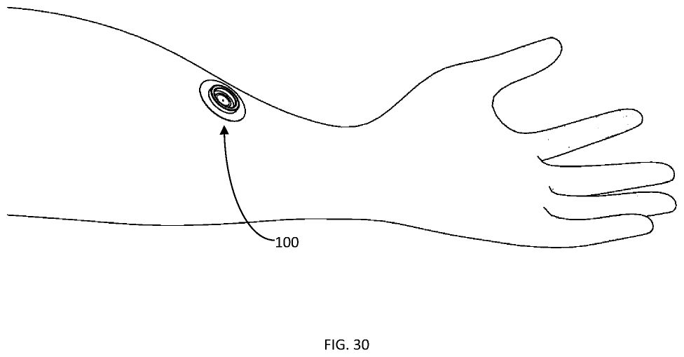

FIG. 31 is a perspective view of the installation of the external storage device of FIG. 26 onto a patient, with the external storage device illustrated in this instance installed on the patients arm.

FIG. 32 is an exploded front cross sectional view of to a third embodiment of an access port.

### DETAILED DESCRIPTION

The following detailed description is in reference to the drawings, in which like elements are consistently numbered. The embodiments illustrated in the drawings, which are not necessarily to scale, are not intended to be exhaustive or to limit the invention to the precise form disclosed. The detailed description illustrates non-limiting examples of the principles of the invention.

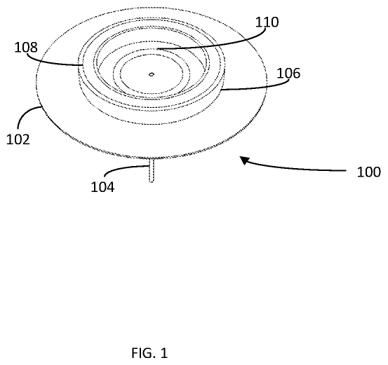

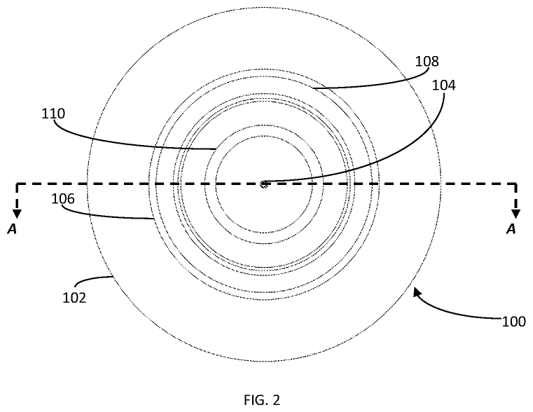

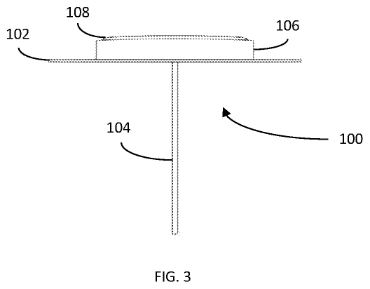

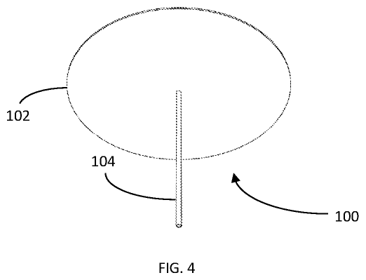

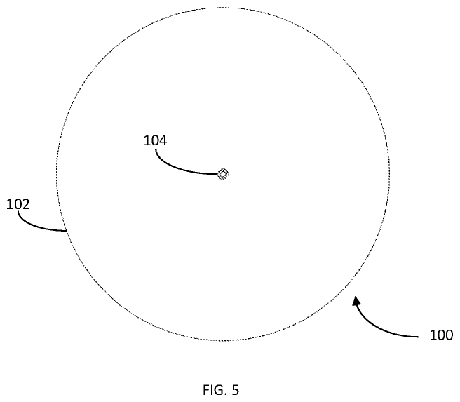

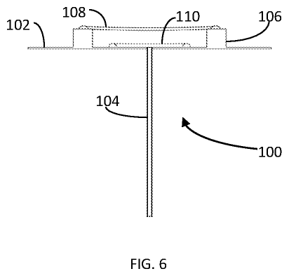

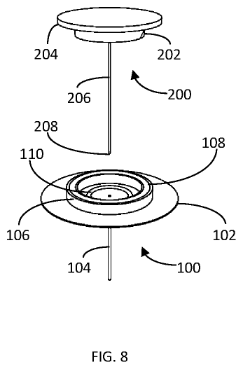

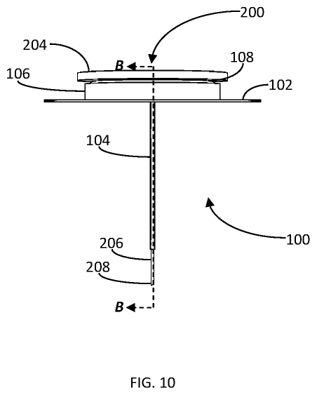

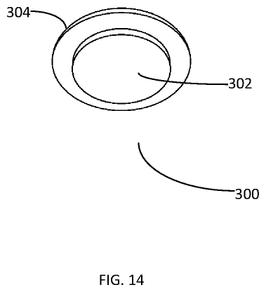

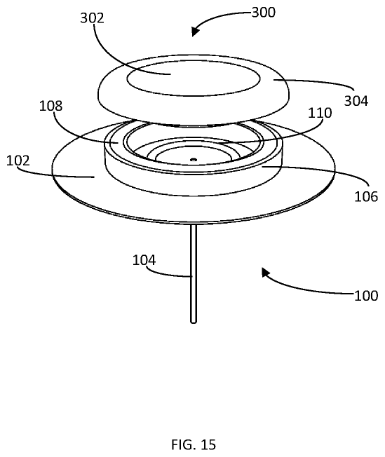

Access port 100 of FIGS. 1 to 6, FIGS. 8 to 11, and FIGS. 15 to 17 includes flanged base 102, with a cannula tube 104 that provides a passageway through the base 102. Chamber wall 106 provides structure to engage the parallel double o-ring sealing mechanism, the mechanism in which the outer o-ring seal 108 works in parallel with inner o-ring seal 110 to assure an air tight connection.

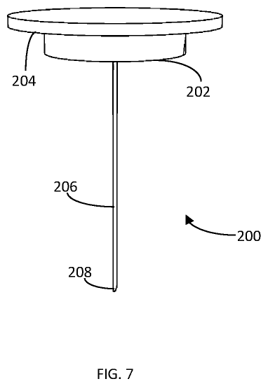

As shown in FIG. 8, access port 100 may be used in conjunction with guide needle 200, of FIGS. 7 to 11, to enhance venous installation. Guide needle 200 of FIGS. 7 to 11 includes a base with a wide portion 204 that engages the outer seal 108 and allows the operator a gripping feature. The base also features a narrow portion 202 that engages the inner seal 110 and the chamber wall 106 which keeps guide needle 200 secured to access port 100 during installation. Guide needle 200 has a needle stem 206 and needle head 208 that has a diameter less than the inner diameter of cannula tube 104.

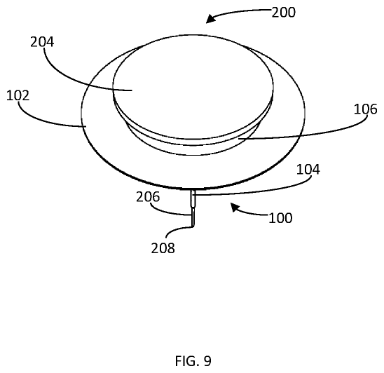

As shown in FIG. 10, when access port 100 and guide needle 200 are assembled, the length of the needle stem 206 exceeds that of cannula tube 104 such that needle head 208 has ample distance to perform its penetrative function. In the venous installation of the assembly of access port 100 and guide needle 200, needle head 208 is used to penetrate the skin and then the targeted vein. Access port 100 is then secured to the patient's body which may be done utilizing adhesives or bandages with the flanged base 102. Once secured, guide needle 200 is pulled away from access port 100 utilizing the finger hold provided from base 204.

With the assembly shown in FIG. 10, access port 100 may be of any material, so long as the needle shaft 206 and needle head 208 are of a material suitable for penetrating skin. For example, flanged base 102 may be of a pliable material or a rigid material depending on the condition of the skin it is to be joined with.

The guide needle 200 may be used to install the access port 100 into a patient's vein. When the access port 100 is assembled with the guide needle, the needle head 208 may be inserted into a patient's veins from a caregiver. When the needle head 208 is in place an adhesive may be placed on the patient's skin or on the flanged base 102. Bandages may additionally be used. The access port 102 is pressed down against the patient's body securing it to the patient. Once secure, the needle 200 is removed by pulling it in reverse using the wide portion of the needle 204 for grip.

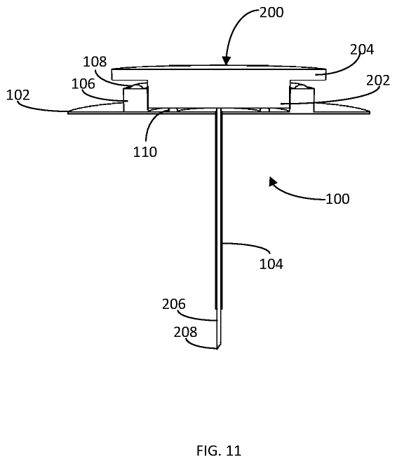

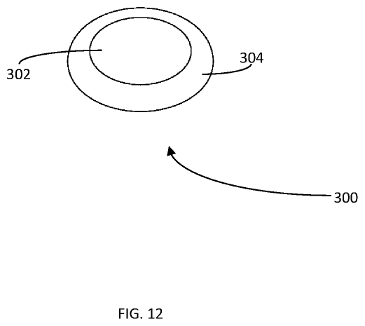

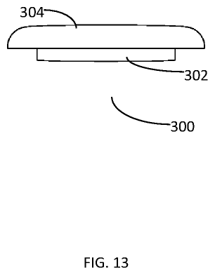

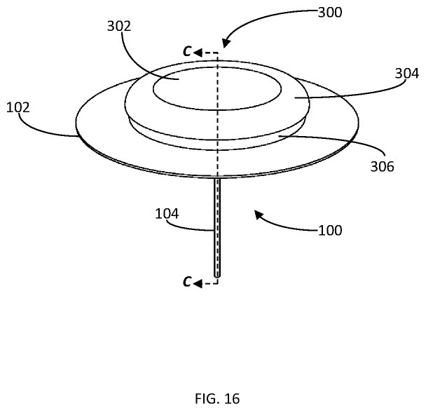

The access port 100 may also be assembled with a sanitary seal 300, of FIGS. 12 to 17, which enables long term installation. The seal 300 consists of an inner seal 302 that engages the inner o-ring seal 110, and an outer seal 304 that engages the outer o-ring seal 108. The chamber wall 106 further engages both the inner 302 and outer 304 sanitary seal to secure the sanitary seal 300 in place. The diameter of the inner seal 302 does not exceed that of the inner diameter of the chamber wall 106. The outer seal 304 overhangs the chamber wall 106 and has rounded edges so that the assembly has a smooth profile for enhanced ergonomics. In FIG. 17, a cross-sectional view of the assembly shows two embodiments of how the sanitary seal may engage an o-ring seal. The inner seal 302 has a flat surface that engages and presses downward on the inner o-ring seal 110. The outer seal 304 is molded to the form of the o-ring 108 so that the seal presses downward on the chamber wall 106 as well as the o-ring 108.

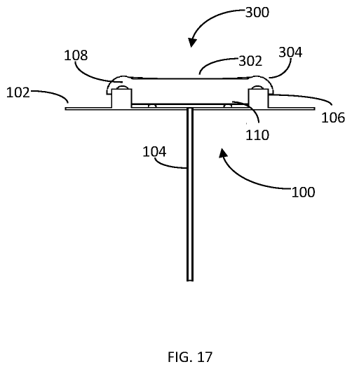

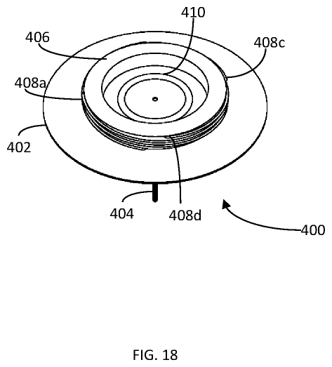

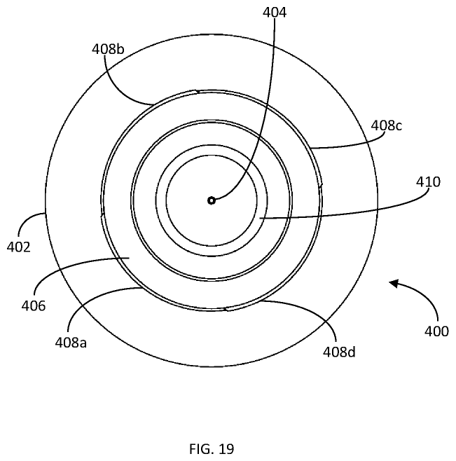

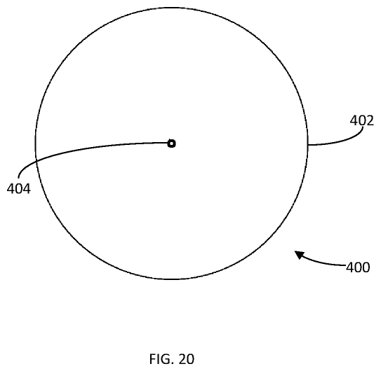

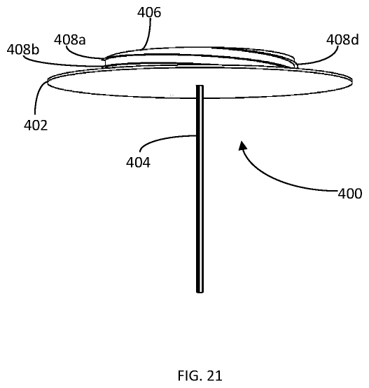

The access port 400, of FIGS. 18 to 22 includes, flanged base 402 from which chamber wall 406 protrudes away from the patient, and cannula tube 404 which provides access into the patient's body. In this embodiment of the access port, a double seal mechanism utilizes an outer threaded seal 408a-b which works in parallel with the inner o-ring seal 410.

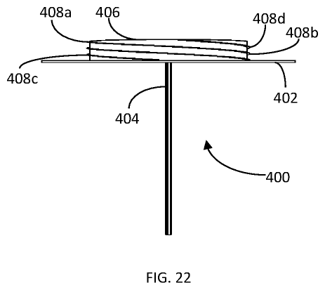

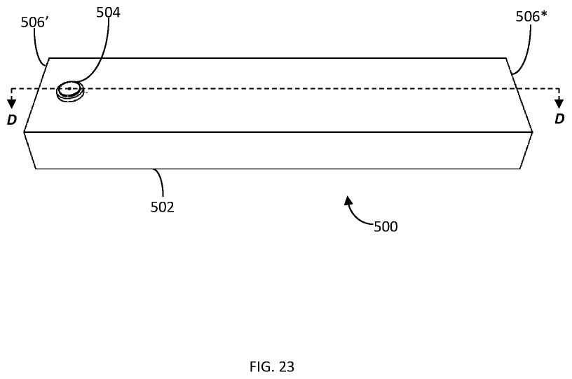

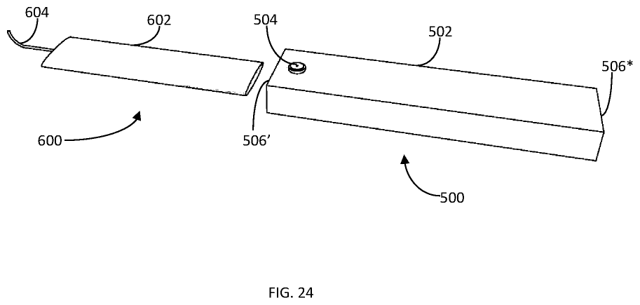

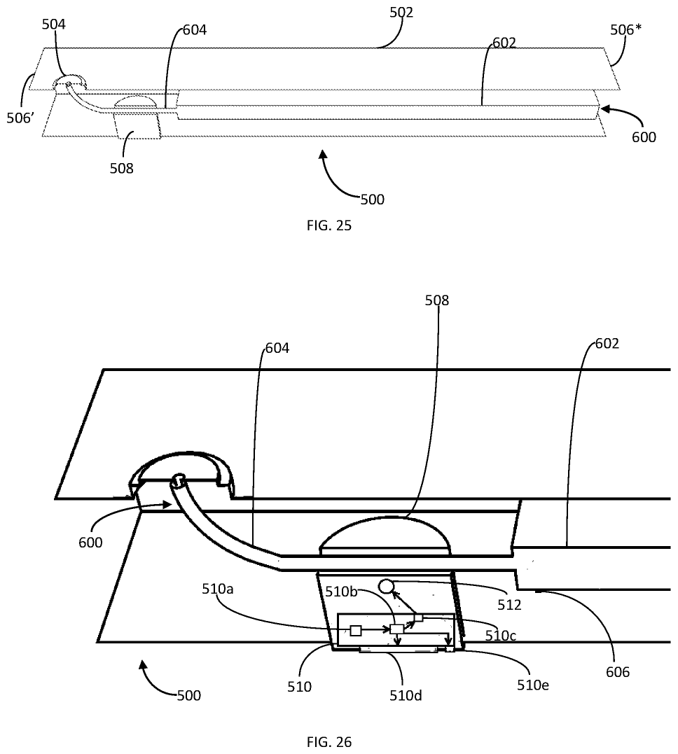

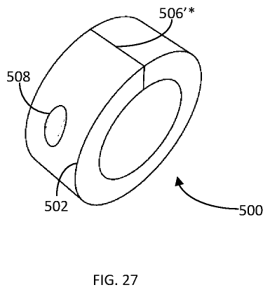

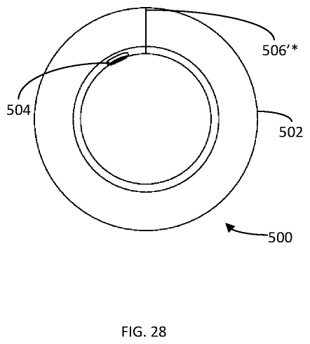

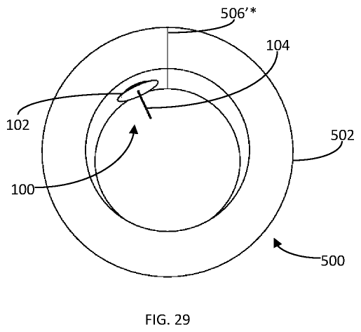

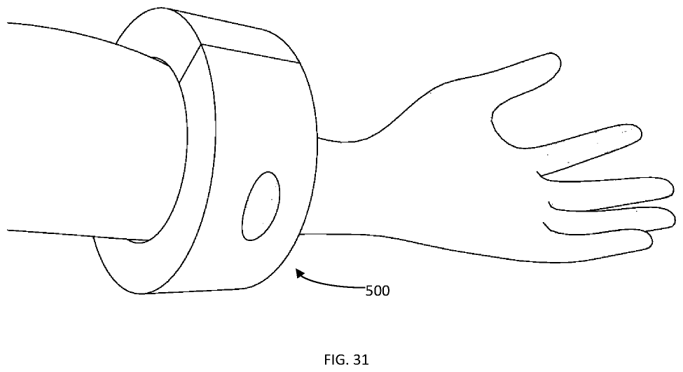

External storage device 500, of FIGS. 23 to 29, and FIG. 31, is one embodiment of a device for delivering substances through the access port 100. The adaptor 504 engages both the outer 108 and inner 110 o-ring seals. To deliver a substance, first the near end 506′ is disengaged from the far end 506*. Then a substance container 600, of FIGS. 24 to 25, is placed inside the body of the device 502 from the near end 506′. The body of the container 602 is placed on the distal side towards the far end 506*. The flow tube 604 is locked into the peristaltic pump 508 and then the end of the tube is inserted into the adaptor 504. Once the flow tube 604 is secured the adaptor may be inserted into the chamber wall 106 of the access port, thereby engaging the double seal. The near end 506′ may then be fastened to far end 506* forming fastened portion 506′*. In this embodiment the fastening is performed with touch fasteners, however the means of fastening is not intended to be limited. Programmable component 510 and mechanical component 512 are housed inside pump 508. The programmable component receives a signal through communication subcomponent 510a. In this embodiment the signal is generated from programmable component 606 located on the substance container 600. The signal may be generated through means of WiFi, near-field communication, radio-frequency, or other means of identification technology. The signal is then sent to data processing subcomponent 510b which then relays the signal to several proceeding subcomponents. The control subcomponent 510c can set the power metering and pump speed to the mechanical component 512 otherwise known as the pump head. The pump may be disabled until the appropriate signal is received whereupon the pump is powered on. A signal may be sent to display screen 510d which will provide the user information such as instructions, system status and validation, identity of the substance in place, estimated time remaining, and other such information. The external communication subcomponent 510e may send and receive signals from remote sources. The data from the system may be analyzed remotely to help optimize subsequent doses. It may also be used to verify patient participation and dose regime. For doses that are intended for mobile use, a GPS compatible version of the external communication subcomponent 510e will enable the ability scientific studies concerning the viability of infusions with mobile patients. Furthermore, new protocols for allowable substances may be input to the system through the external communication subcomponent 510e.

The programmable component enables for remote monitoring of a patient's dosage regime. For a therapeutic substance, this will allow a caregiver or doctor to make sure that the patient does not have a lapse in treatment. If any lapses or lack of participation does occur the exact duration of that period may be accurately tracked. Additionally, the programmable features make the Band and Bag system user friendly by removing the need for the patient themselves to change the settings of the device. The settings may be set by a doctor, caregiver, or manufacturer. Minimizing the manipulation that a patient has to perform is particularly necessary for younger patients.

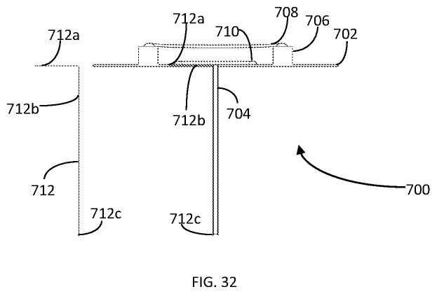

The access port 700 of FIG. 32 includes flanged base 702, with a cannula tube 704 that provides a passageway through the base 702. Chamber wall 706 provides structure to engage the parallel double o-ring sealing mechanism, the mechanism in which the outer o-ring seal 708 works in parallel with inner o-ring seal 710 to assure an air tight connection. The access port also features sensor 712 which enables the access port to monitor a patient's blood pressure, heart rate, and biological markers. The sensor has a programmable component 712a which is located between outside of the inner seal 710. The programmable component 712a may export information to external storage device 500 for further processing. Wire 712b is nested inside of the device, running through the flanged base 702 and through the cannula 704 where it connects the sensor head 712c to the programmable component 712a. The wire 712b transmits the signal from the sensor as well as power to the sensor if necessary. The power source may come from an external connection, for example, from contact with the adaptor 504 of external storage device 500. The ability to detect a patient's vital signs enhances the interaction between a patient and a caregiver or doctor by producing a high-resolution chart of the patient's response to a dose.

### CONCLUSION, RAMIFICATION, AND SCOPE

Thus the reader will see that at least one embodiment of the venous access port can provide the sterility, ergonomics, user experience, comfort, simplification, and reduced bulk that will improve the patient experience and allow more users to participate in therapies that were previously highly taxing.

1. An access port for transporting fluids into a patient's body, comprising:
A flanged base;
A receptacle with a multitude of seals, the sealing mechanism including but not limited to O-rings, screw-locks, septa, and friction seals;
A cannula secured to the base and establishes a flow path between the receptacle and patient's body;
A passageway perpendicular to the plane of the flanged base that establishes a flow path from the receptacle to the cannula

2. The access port of claim 1 wherein the flanged base is adhered or bandaged on the patient's skin to secure the access port in place.

3. The access port of claim 1 wherein the flanged base is implanted subcutaneously to secure the access port to the patient's body

4. The access port of claim 1 wherein the receptacle is defined as being multiply sealed with sealing mechanisms utilizing a variety of sealing mechanisms or a multiplicity of one or more sealing mechanism, with at least one pair of seals in parallel.

5. The access port of claim 1 wherein the receptacle is capped after installation with a sanitary adaptor which engages more than one seal of the receptacle.

6. The access port of claim 1 wherein the receptacle is capped after installation with an adaptor which engages more than one seal of the receptacle and is connected to an external device.

7. The access port of claim 1 wherein the cannula is installed intravenously using a guide needle, the guide needle engaging a multitude of seals of the receptacle, and the guide needle being removed after installation.

8. The access port of claim 7 wherein the cannula is comprised of a flexible material.

9. The access port of claim 1 wherein the cannula is installed subcutaneously, the cannula being comprised of a rigid material sufficient to penetrate the patient's skin, and the cannula being of sufficient length such that the flanged base may be secured to the patient while the cannula provides subcutaneous access to the patient.

10. The access port of claim 1 wherein the passageway allows unidirectional flow from an external source that engages more than one seal in the receptacle into the patient.

11. The access port of claim 1 wherein the passageway allows bidirectional flow between an external source that engages more than one seal in the receptacle and the patient.

12. The access port of claim 1 wherein the base and receptacle are comprised of a material compatible to being secured to a patient's body by means of, but not limited to, adhesives or bandages.

13. The access port of claim 1 wherein the access port is not consumed or deteriorated in instances of fluid transfer and may remain installed on the patient for a multitude of fluid transfer instances.

14. The access port of claim 1 further comprising sensors located in the cannula, the sensors including but not limited to heart rate and blood pressure, which may monitor a patient's vital signs and biological markers.

15. An access port for transporting substances across the patient's skin, that may remain installed for a multitude of substance transfer instances, comprising of a multiply sealed mechanism with at least one seal functioning in parallel to another seal.

16. The access port of claim 15 wherein one or more sealing mechanism may include, but is not limited to, o-ring, friction, screw lock, magnetic, or septa seals.

17. The access port of claim 15 further comprising an adaptor that engages the multiply sealed mechanism securing a flow path to an external source.

18. The access port of claim 17 wherein the adaptor engages more than one seal of the access port at one end and the other end is fit to external devices including but not limited to infusion pumps, filters, tubing, and connectors.

19. An external storage device worn by a patient that houses a substance and facilitates the delivery of the housed substance into the patient's body comprising of a parallel multiply sealed adaptor.

20. The external storage device of claim 19 further including an external source that is an arm band comprising:
A flexible hollow casing;
Two ends that may be fastened together with making the device loop shaped with adjustable diameter;
A pump.

21. The external storage device of claim 20 wherein the pump contains a programmable component comprising:
A data processing subcomponent;
A communication subcomponent that relays information to the data processing subcomponent;
A control subcomponent that meters the power to the mechanical components of the pump;
An external communication subcomponent;
A display screen.

22. The external storage device of claim 20 further comprising a storage container that fits inside the hollow portion of the arm band, comprising:
A flexible body that contains a substance;
A flow tube through which substances may leave the storage container.

23. The storage container of claim 22 further comprising a unique identification component that specifies the identity and volume of the substance contained within, the identification component comprising one or more technologies including but not limited to radio-frequency identification near-field communication, WiFi, visual sensors, or physical sensors.

24. A substance container for materials that are desired to be transported into a patient's body, comprising:
A body to house the substance;
A flow tube that wherein substance is transported out from the container;
An adaptor that engages more than one seal.

25. The substance container of claim 24 wherein the flow tube is attached to the body at one end, and the other end of the flow tube is attached to the adaptor.

26. A programmable component that may be embedded into a storage device, substance container, or access port that identifies substances such that system will only function with allowed substances, a programmable component comprising one or more of:
A data processing subcomponent;
A communication subcomponent that relays information to the data processing subcomponent;
A control subcomponent that meters power to other components of the device such as mechanical components;
An external communication subcomponent;
A display screen.

27. The programmable component of claim 26 wherein a substance container comprises only of a communication subcomponent, a communication subcomponent with a capacity to utilize identification technology including but not limited to radio frequency, near-field communication, visual sensors, and physical sensors.

28. The programmable component of claim 26 wherein a storage device is equipped with a communication and a control subcomponent that respond to an identification signal by metering power to other components of the storage device.

29. The programmable component of claim 28 wherein the power metering response may originate from an identification signal generated the programmable component of a substance container.

30. The programmable component of claim 26 further comprising connecting to a sensor for monitoring patient response including but not limited to heart rate, blood pressure, and biomarker levels.

31. The programmable component of claim 26 wherein the collected data is sent to a remote storage location for further data analysis which may be used to optimize the configuration for subsequent uses.

32. The programmable component of claim 26 further comprising the capacity receive communication from remote sources which modifies the operational parameters of the system to meter power to mechanical components.

33. The programmable component of claim 26 wherein the external communication subcomponent is equipped with global positioning system (GPS) capacity.

34. An automated substance recognition method, comprising:
Receiving input from a substance container to specify the operational parameters of the transferal device;
Configuring power metering to mechanical components of the transferal device to either allow or disallow mechanical function based on the substance container input received;
Specifying a period of time for operation of mechanical functions;
Dispensing substances into a patient's body;
Collecting data of patient response and system operation then exporting that data to a remote location.

35. The method of claim 34 wherein the operational parameters further comprise of one or more of flow rate, pump head speed, or pressure.

36. The method of claim 34 wherein the exported data provides feedback to doctors or caregivers for subsequent dosages.

37. The method of claim 34 wherein the substance is delivered subcutaneously into the patient's body.

38. The method of claim 34 wherein the substance is delivered intravenously into the patient's body.

39. The method of claim 34 wherein the substance container is housed within the transferal device.

40. The method of claim 39 wherein the transferal device is worn on the patient's body.

41. The method of claim 34 wherein the transferal device contains a small-scale pump as a mechanical component which enables the transfer of fluids from the substance container into the patient's body.

42. The method of claim 34 wherein substances are dispensed along a flow-path established between the patient's body and the substance container using an access port.
